(19) United States
(12) Patent Application Publication
(10) Pub. No.: US 2016/0310529 A1
(43) Pub. Date: Oct. 27, 2016

(54) NUTRITION, EXERCISE AND COGNITIVE DEVELOPMENT

(71) Applicant: NESTEC S.A., Vevey (CH)

(72) Inventors: Nora Schneider, Epalinges (CH); Gertrude Gentile-Rapinett, Jongny (CH); Khurram Zia, Corseaux (CH); Ellen Grace Isturis, Makati City (PH); David Stephane Philippe, Lausanne 25 (CH)

(21) Appl. No.: 15/103,424

(22) PCT Filed: Dec. 10, 2014
(86) PCT No.: PCT/EP2014/077298

§ 371 Date: Jun. 10, 2016

(30) Foreign Application Priority Data
Dec. 10, 2013 (EP) 13196375.3

(51) Int. Cl. A61K 33/30; A61K 33/06; A61K 31/51; A61K 31/513; G09B 19/00; A61K 31/047; A61K 31/375; A61K 31/202; A61K 9/00; G09B 5/02; A61K 33/26; A61K 31/675

(52) U.S. Cl. CPC A61K 33/30; A61K 33/26; A61K 33/06; A61K 31/51; A61K 31/513; A61K 31/675; A61K 31/047; A61K 31/375; A61K 31/202; A61K 9/0095; A61K 9/0053; G09B 5/02; G09B 19/003; G09B 19/00; A23L 33/40; A23L 33/30; A23L 33/16; A23L 33/15; A23L 33/12; A23V 2002/00

(57) ABSTRACT

The present invention relates generally to supporting the optimal development of children through nutrition and cognitive exercise. In particular, the present invention relates to a growing-up milk composition comprising per daily dose at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, at least 0.15 g Alpha Linolenic Acid and to its use in the prevention of a delayed brain and cognitive development in children in combination with a cognitive exercise program.

The present invention relates generally to supporting the optimal development of children through nutrition and cognitive exercise. In particular, the present invention relates to a growing-up milk composition comprising per serving at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, at least 0.15 g Alpha Linolenic Acid and to its use in the prevention of impaired and/or suboptimal brain functioning and delayed cognitive development in children in combination with a cognitive exercise program.

It is well known today that nutrition plays an important role in brain functioning and cognitive development of children. Dietary deficiencies, e.g., in some minerals, vitamins or essential fatty acids can lead to alterations of brain functions and subsequently in cognitive development.

Early childhood is often described as the most important phase for overall development throughout the lifespan. Scientists suggest that the brain and biological development during the first years of life is also influenced by an infant's environment. Early experiences appear to determine health, education and economic participation for the rest of life. Notably, every year, more than 200 million children under five years old fail to reach their full cognitive and social potential (WHO Fact Sheet 332). Several risk factors appear to affect at least 20-25% of infants and young children, e.g., in developing countries: malnutrition that is chronic and severe enough to cause growth stunting, inadequate stimulation or learning opportunities, and mineral deficiency. Scientists have therefore focused on providing nutritional compositions that specifically satisfy the developmental needs of children.

With respect to mineral and vitamin supplementation, several studies showed improvement in some domains of brain function. Iron supplementation, for example, improved significantly the cognitive function in anemic children older than 2 years. The European Food Safety Authority (EFSA) has so far released several favorable opinions on the role of vitamins and minerals, e.g., for iodine, magnesium, thiamin (vitamin B1), niacin (vitamin B3), pyridoxine (vitamin B6), biotin (vitamin B8) and vitamin C, in brain function provided that certain requirements are met.

With respect to fatty acids, for example, docosahexaenoic acid (DHA), alpha-linolenic acid (ALA) and linoleic acid (LA), the EFSA has issued a positive opinion on DHA and its contribution to the maintenance of normal brain function, on ALA and its contribution to brain development, and on essential fatty acids in general and their contribution to normal growth and development of children, provided that certain requirements are met. Polyunsaturated fatty are also described in the literature as contributors to an improved cognitive performance and psychosocial functioning of children.

Research further shows that deficiencies in mental stimulation, and in the quality of the caring relationship experienced by the child, may stunt its emotional, social, physical and cognitive development (WHO (1999) A Critical Link: Interventions for physical growth and psychological development: A Review). In line with this, Warsito et al., find the lowest cognitive development in children with low psychostimulation (Warsito O et al., 2012, Nutrition Research and Practice 6(5); 451-457).

The present inventors therefore conclude that not only optimal nutrition but also optimal cognitive stimulation during childhood are essential measures in the treatment or prevention of a delayed brain development in children.

It would therefore be desirable to have a holistic solution available that allows to best support cognitive development.

Any reference to prior art documents in this specification is not to be considered an admission that such prior art is widely known or forms part of the common general knowledge in the field.

The objective of the present invention was, hence, to improve the state of the art and in particular to provide a solution to treat or prevent a delayed cognitive development in children that considers the multi-factorial and multi-dimensional nature of the brain development process, or to at least provide a useful alternative.

The inventors were surprised to see that the object of the present invention could be achieved by the subject matter of the independent claims. The dependent claims further develop the idea of the present invention.

In particular, the inventors show that the administration of a specific nutritional composition together with carrying out a cognitive exercise program was able to improve the cognitive performance of children and/or was able to decrease delayed cognitive development in children.

The cognitive exercise program comprises tasks aimed at improving language skills, memory, attention, problem solving skills and/or psychomotor skills.

Notably, the positive effect is shown in children from an average population and not in children with known predominant nutritional deficiencies that have a negative effect on cognitive performance.

For example, the administration of the specific nutritional composition of the present invention together with carrying out the cognitive exercise program exercise program of the present invention was in particular effective in children with nutritional deficiencies and/or in children that live in environments with low cognitive stimulation, for example resulting from very limited access to educational material such as books or toys and/or as a result of absence of dedicated and quality interactions with caregivers The quality and quantity of stimulation in the home environment is typically assessed in the art with the Home Observation for Measurement of the Environment (HOME) inventory. This tool is well accepted, publically available and can be obtained from the ASU, School of Social and Family Dynamics, Cowden Family Resources, 850 S. Cady Mall |PO Box 873701, Tempe, Ariz. 85287-3701, USA (http://fhdri.clas.asu.edu/home/contact.html).

Higher total HOME scores indicate a more enriched home environment. In general, scores falling in the lowest fourth of the score range indicate and environment that may pose a risk to some aspect of the child's development” (Totsika & Sylva (2004), Child and Adolescent Mental Health, Vol. 9(1), 25-35).

The total score can range from 0 to 55. “Low cognitive stimulation” may be for the purpose of the present invention a score of 15 or less using the HOME inventory.

The present invention provides a composition comprising per serving at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, and at least 0.15 g Alpha Linolenic Acid in combination with a cognitive exercise program comprising tasks to be carried out at least once a week, each time for at least 10 minutes, for use in the prevention or treatment of a delayed brain and cognitive development in children.

The composition of the present invention in combination with the cognitive exercise program of the present invention may also be used to support the prevention or treatment of a delayed brain and cognitive development in children.

The present invention also relates to a composition comprising per serving at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, and at least 0.15 g Alpha Linolenic Acid.

The composition ideally contains milk or may be a milk based composition. For example, it may be provided in a powdered form to be reconstituted in water to ensure that the composition can easily be stored and does not need refrigeration.

As used in this specification, the words “comprises”, “comprising”, and similar words, are not to be interpreted in an exclusive or exhaustive sense. In other words, they are intended to mean “including, but not limited to”.

Consequently the present invention relates in part to a composition comprising per serving at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, at least 0.15 g Alpha Linolenic Acid, in combination with a cognitive exercise program comprising tasks stimulating language skills, memory, attention, problem-solving skills and/or psychomotor skills to be carried out at least 3×/week, each time for at least 10 minutes, for use in the prevention or treatment of a delayed brain and cognitive development in children.

The present invention also relates to the use of a composition comprising per serving at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, at least 0.15 g Alpha Linolenic Acid in combination with a cognitive exercise program comprising tasks stimulating language skills, memory, attention, problem-solving skills and/or psychomotor skills to be carried out at least 3×/week, each time for at least 10 minutes, in the prevention or treatment of a delayed brain and cognitive development in children.

The composition may contain at least 4.4 mg Zinc, at least 4.0 mg Iron, at least 80 mg Magnesium, at least 0.4 mg Vitamin B1, at least 5.2 mg Vitamin B3, at least 0.48 mg Vitamin B6, at least 8 μg Vitamin B8, at least 24.8 mg Vitamin C, and at least 0.28 g Alpha Linolenic Acid per daily dose.

The daily dose may typically contain 2 servings. A serving may be 100 ml, 150 ml, 200 ml, 250 ml or 330 ml, for example.

The composition may be provided in liquid or in powdered form, to be reconstituted in milk or in water. Alternatively, the composition may already contain milk or may be a milk based composition.

For example, a serving may be prepared by adding 36 g of the powdered composition to 225 ml water. Alternatively, a serving may be prepared by adding 26 g of the powdered composition to 180 ml water.

The composition may contain, for example, per 100 g dry weight 19-21 g fat, e.g., 19.5-20.5 g fat; 10-21 g protein, e.g., 19.5-20.5 g protein; and 47-51 g carbohydrates, e.g., 48-50 g carbohydrates. The composition may be a milk based composition. It may further contain per 100 g dry weight 30-36 mg DHA, for example 32-34 mg DHA.

As such, the composition may have a caloric content of 400-500 kcal per 100 g dry weight, for example of 450-470 kcal per 100 g dry weight.

The cognitive exercise program may comprise tasks stimulating at least one, at least two or at least three cognitive domains, such as language skills, memory, attention, problem solving skills and/or psychomotor skills.

For example, the cognitive exercise program may contain tasks stimulating language skills, memory, and attention.

Alternatively, the cognitive exercise program may contain tasks stimulating language skills, memory, and problem solving skills.

Further alternatively, the cognitive exercise program may contain tasks stimulating memory and problem solving skills.

The task may be to be carried out at least once a week, at least twice a week, at least three times a week or daily.

Each task may be to be carried out for at least 2 minutes, each, at least 3 minutes each, at least 5 minutes each or at least 10 minutes each. The combined cognitive exercise program may be to be carried out for at least 5 minutes, at least 10 minutes, at least 15 minutes or at least 20 minutes.

For the purposes of the present invention, children are between 1 and 14 years old, for example between 2 and 8 years old. In accordance with the clinical study carried out by the inventors children may be between 3 and 5 years old.

The children may be—but do not have to be—malnourished. Alternatively and/or additionally, the children may be moderately or low stimulated in their home environment. The degree of stimulation can be measured by the HOME Inventory. The Home Observation Measurement of the Environment (HOME) is the primary measure of the quality of a child's home environment and has been extensively used as both an input in helping to explain other child characteristics or behaviors and as an outcome in its own right for researchers whose objective is to explain associations between the quality of a child's home environment and earlier familial and maternal traits and behaviors. The HOME inventory (Bradley, R. H. & Caldwell, B. M., 1984, Developmental Psychology, 20) is well established as a unique observational measure of the quality of the cognitive stimulation and emotional support provided by a child's family.

The positive effect of the combined administration of the composition and cognitive exercise program, for example on prevention or treatment of a delayed cognitive development in children can be measured as using the Wechsler Pre-School and Primary Scale of Intelligence, 4th edition (WPPSI-IV), its content is hereby incorporated herein by reference. This is a test aimed at measuring overall cognitive abilities in children between 2 and 7 years. The results are expressed, for example as
Verbal Comprehension Index (VCI)
Visual Spatial Index (VSI)
Working Memory Index (WMI)
Fluid Reasoning Index (FRI)
Processing Speed Index (PSI)
Vocabulary Acquisition Index (VAI)
Nonverbal Index (NVI)
General Ability Index (CAI)
Cognitive Proficiency Index (CPI)

Consequently, the combined administration of the composition and cognitive exercise program in accordance with the present invention may be for use in improving the Verbal Comprehension Index (VCI), the Visual Spatial Index (VSI), the Working Memory Index (WMI), the Fluid Reasoning Index (FRI), the Processing Speed Index (PSI), the Vocabulary Acquisition Index (VAI), the Nonverbal Index (NVI), the General Ability Index (CAI), and/or the Cognitive Proficiency Index (CPI).

The inventors find that the cognitive exercise program is more effective if it is done together with at least one other family member. A functioning and warm relationship appears to have a positive effect on the beneficial effect of the program.

Particularly, in the area of attention, selective and/or sustained attention can be improved significantly. Tasks that support this area of cognition include for example searching for a hidden target object and/or finding the matching object.

In the area of language, in particular speech, comprehension, and/or reading can be improved. Tasks that support this area of cognition include reading interactively a story and highlighting interesting and relevant characteristics in the story.

In the area of memory, in particular short term memory, long term memory, things memory and/or event memory can be improved. Tasks that support this area of cognition include remembering presented visual material and recognizing what matches or complements the previously presented information.

In the area of problem solving, in particular executive functions, reasoning, maths concepts and/or multitasking can be improved. Tasks that support this area of cognition include seeing how smaller objects fit together as part of a whole, finding solutions to tasks numerically, spatially, linguistically.

In the area of psychomotor skills, in particular gross motor and/or fine motor skills can be improved. Tasks that support this area of cognition include tasks that elicit finger dexterity, movements of the hands and fingers to achieve a goal.

The exercise program may be to be carried out together with at least one other family member. This at least one other family member may be the father, the mother or a grandparent. For example, the cognitive exercise program is to be carried out together with the mother.

The cognitive exercise program may be provided on paper. For example, possible cognitive exercises may be printed on the packaging of the nutritional composition. The inside of the pack may be used for this purpose. Additional or alternatively, a leaflet with corresponding cognitive tasks may be provided, for example, inside the pack of the nutritional composition.

Additionally and/or alternatively, the cognitive exercise program may be provided as an application for a computational device, such as a smart phone, a computer, and/or a tablet PC. Using a computational device has many advantages. For example, children are generally attracted to these devices, so that it is more likely that they will spend more time carrying out the exercises when they are provided on a computational platform.

Further, games can be re-played and they can better be shared with friends.

To measure the progress a child it is one embodiment of the present invention that the performance of the child is scored and the scores are recorded. This allows it, for example, to compare present results to previous results and to show the learning progress of a child. This can serve as motivation. Hence, in one embodiment of the present invention, the performance of the child in the tasks is compared to its previous performances.

If the exercise program is provided as an application for a computational device, the performance of the child in the tasks may be displayed on screen and compared to previous performances.

Every child is developing at its own speed, and hence it is preferred to compare the child's present performance with its previous performances.

Still, for example for research purposes, it may be useful to compare the child's performance with the performance of comparable children. This allows to determine and above average, average, or below average performance, for example.

For example, using a computational platform it is possible to build a database of the performances of children, for example, of comparable age, and to compare individual results with the resulting average performance. This also allows monitoring the improvement of the performance of a child during the exercise program in relation to the average of comparable children.

Optimal nutritional support is essential for ideal cognitive development. Hence, the composition of the present invention may be to be consumed at least once per day. For example, the composition of the present invention may be to be consumed at least twice per day.

To ensure optimal support during the day, the composition may be to be consumed in the morning, for example as part of a breakfast. A second portion may be to be consumed during a break at school or at lunch time or as part of a dinner, for example.

To make sure that the nutritional level is ideal when the cognitive training program is carried out it is advisable if the nutritional composition is consumed at most 6 hours, for example at most 4 hours before the exercise is carried out.

For example, the consumption of the composition and the execution of the exercise program are no more than 3 hours apart.

The inventors have found that the specific nutritional composition of the present invention and the specific cognitive exercise program described herein—if combined—act synergistically and can deliver a number of important health benefits.

For example, the composition and the cognitive exercise program for use in accordance with the present invention may be for use in improving the cognitive abilities of children. Improved cognitive abilities will result in a faster mastering of the challenges of daily life and a more enjoyable school experience.

The composition and cognitive exercise program for use in accordance with the present invention may be, for example, for use in improving the learning abilities of children. A faster learning progress can be a significant motivation for children when they discover the world and explore its opportunities.

The composition and cognitive exercise program for use in accordance with the present invention may additionally or alternatively be, for example for use in supporting brain development in children and/or for use in ensuring normal brain function in children.

As such, the subject matter of the present invention is relevant for malnourished and/or under-stimulated but also for normal children in good health that grow up in stable social relationships.

The present invention further provides a nutritional composition. The nutritional composition has the advantage that it can be used for all the purposes mentioned above, for example in the prevention or treatment of a delayed brain and cognitive development in children. The nutritional composition effective on its own, but in particular, if it is combined with the cognitive exercise program described above.

Accordingly, the present invention provides a composition comprising per daily dose at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, and at least 0.15 g Alpha Linolenic Acid.

The composition may for example be provided on a milk basis or as milk based beverage, both, as a ready-to-drink format or as a dry preparation. Such a dry preparation may for example be powdered.

Powdered compositions in accordance with the present invention may be milk based, so that they can be reconstituted in water to yield a milk-based beverage.

For example, one serving of the composition of the present invention may contain

| Nutrient (unit) | Minimum amount per serving |
|---|---|
| Zinc (mg) | 2.25 |
| Iodine (μg) | 22.5 |
| Iron (mg) | 2.1 |
| Mg (mg) | 45 |
| Thiamin (B1) (mg) | 0.21 |
| Niacin (B3)(mg) | 2.7 |
| Pyridoxin (B6)(mg) | 0.3 |
| Biotin (B8) (μg) | 4.5 |
| Vitamin C (mg) | 12.6 |
| EPA + DHA (mg) | 75 |
| α-Linolenic ac. (ALA)(g) | 0.161 |
| Linoleic acid (LA)(g) | 2.05 |

If provided as a dry milk based formulation, the composition of the present invention may comprise per 100 g:

| Nutrient (unit) | Amount |
|---|---|
| Fats (g) | 19-23 |
| Protein (g) | 14-21 |
| Carbohydrates (g) | 48-50 |
| Zinc (mg) | 6.0-6.5 |
| Iodine (μg) | 65-71 |
| Iron (mg) | 7.6-8.0 |
| Mg (mg) | 115-135 |
| Thiamin (B1) (mg) | 0.5-0.8 |
| Pyridoxin (B6)(mg) | 1.0-1.2 |
| Biotin (B8) (μg) | 1.1-1.4 |
| DHA (mg) | 15-20 |
| α-Linolenic ac. (ALA)(mg) | 420-600 |

Those skilled in the art will understand that they can freely combine all features of the present invention disclosed herein. In particular, features described for the product of the present invention may be combined with the use of the present invention and vice versa. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### EXAMPLE

### Clinical Study for Assessing General Cognitive Abilities in Children 3 to 5 Years of Age

### Study Population:

The study population consists of a total number of 300 enrolled subjects (20% drop out), male or female children aged 3 to 5 years who meet the following inclusion criteria will participate in the study.

### Subject Inclusion Criteria:

Male and female children, age 3;0-4;11 years
Having obtained their legal representative's informed consent
Level of stimulation in their home environment below the mean score of the HOME inventory
Normal cognitive development as screened by the DIKNAS
Normal growth (−2SD to +2SD z-score)

### Intervention:

The study intervention consists of a growing-up milk product in accordance with the present invention that is daily consumed (2×/d; for composition see table below) in combination with cognitive stimulation exercises (iPad games and offline learning activities) over a period of 6 months.

The table below describes the macronutrient composition of the three recipes used for the clinical trial (Serving size: 36 g).

| | | Control | GUM2 with the | GUM1 with the |
|---|---|---|---|---|
| | | product | composition of | composition of |
| Macro- | | (skimmed- | the present | the present |
| nutrients | Unit | milk formula | invention | invention |
| Energy | kcal/100 g | 467.8 | 461.9 | 478.6 |
| | g/serving | 168.4 | 166.3 | 172.3 |
| Fat | g/100 g | 17.8 | 21.2 | 21.4 |
| | g/serving | 6.4 | 7.6 | 7.7 |
| alpha-LA | mg/100 g | 78.0 | 518.0 | 586.0 |
| | mg/serving | 28.1 | 186.5 | 211 |
| Protein | g/100 g | 14.0 | 16.3 | 15.3 |
| | g/serving | 5.0 | 5.9 | 5.5 |

The growing-up milk furthermore contains Zinc, Iron, Mg, Thiamin, Niacin, Pyridoxin, Biotin, Vitamin C and Alpha Linolenic acid.

### Methods:

The tests and methods used to assess efficacy of the intervention include cognitive (WPPSI-IV; ASQ-3), behavioral/mother-child interaction (PICCOLO) and self-rating measures.

1) The Wechsler Pre-School and Primary Scale of Intelligence, 4^{th} edition (WPPSI-IV) is a test for cognitive abilities that consists of primary index scales (Verbal Comprehension Index (VCI), Visual Spatial Index (VSI), Working Memory Index (WMI), Fluid Reasoning Index (FRI), Processing Speed Index (PSI)), a full scale IQ, and ancillary index scales (Vocabulary Acquisition Index (VAI), Nonverbal Index (NVI), General Ability Index (CAI), Cognitive Proficiency Index (CPI)). It is a widely used test that is appropriate for children 2 to 7 years of age.

2) Mother-child-interaction as measured by observation: The interaction is videotaped with at least 10 minute sequences. Interaction is analyzed using an observational measure of positive parenting interactions with very young children, called Parenting Interactions with Children: Checklist of Observations Linked to Outcomes (PICCOLO). This checklist was developed for children up to 3 years; however it has been applied for up to 5 years of age. The developmental parenting behaviors assessed are affection, responsiveness, encouragement and teaching.

3) Parents' rating of child's development as measured by the Ages & Stages Questionnaire: It is a parent-rating questionnaire for different age stages between 2 and 66 months of age to screen the areas of communication, gross motor, fine motor, problem-solving, and personal-social. It has excellent validity and reliability (see, for example, http://agesandstages.com/asq-products/asq-3/asq-3-at-a-glance/).

4) Nutritional status as assessed by anthropometric measures and by urine analysis: Height-for-age, weight-for-height, and weight-for-age will be calculated after obtaining each child's sex, age, weight, and height. Urine samples of the children are taken. Protein status will be assessed by HPLC measure of 3-methylhistidine in the urine (in a single void collected after the 1st morning one) with correction by urinary creatinin value.

5) Nutritional intake: Is assessed by a 24 hour food recall questionnaire.

6) Child Behavior as assessed by the Child Behavior Checklist (CBCL 1½-5): It is a parent-rated scale that obtains information about young children's behaviors, and it is applicable for the age range 1.5-5 years. Items are scored on syndrome scales designated as Emotionally Reactive; Anxious/Depressed; Somatic Complaints; Withdrawn; Attention Problems; Aggressive Behavior, and Sleep Problems.

The tests and questionnaires are adapted to the culture and language. Test assessment are done separately for each child.

### Interpretation of Results:

At the end of the study, the comparison between the groups shows that the group receiving the nutritional composition and cognitive stimulation performs better in the cognitive, behavioral and interaction areas assessed by the tests and questionnaires mentioned above. In particular, in the group receiving the combination of nutritional composition and cognitive stimulation of the present invention, better cognitive and behavioral improvements are yielded.

One or several of the following results are generated:
The results indicate improved cognitive abilities on at least one measure pertaining to attention, learning, comprehension, language, reasoning, memory, information processing, and concentration in children receiving the composition of the present invention.
The results indicate improved caregiver-child interaction on at least one measure pertaining to affection, responsiveness, encouragement and teaching in children receiving the composition of the present invention.
The results indicate improved child development on at least one measure pertaining to communication, gross motor, fine motor, problem-solving, and personal-social domains.
The results indicate improved nutritional status as assessed by anthropometric measures as described above.
The results indicate improved child behavior on at least one domain pertaining to Emotionally Reactive, Anxious/Depressed, Somatic Complaints, Withdrawn, Attention Problems, Aggressive Behavior, and Sleep Problems.

1. A method for use in the prevention or treatment of a delayed cognitive development in children comprising administering a composition comprising per serving at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, at least 0.15 g Alpha Linolenic Acid to a child in need of same in combination with a cognitive exercise program comprising tasks stimulating at least one of language skills, memory, attention, problem solving skills and/or psychomotor skills to be carried out at least once a week, each time for at least 10 minutes.

2. Method in accordance with claim 1, wherein the exercise program is to be carried out at least three times a week.

3. Method in accordance with claim 1, wherein the exercise program is to be carried out together with at least one other family member.

4. Method in accordance with claim 3, wherein the exercise program is to be carried out together with the mother.

5. Method in accordance with claim 1, wherein the exercise program comprises one or more of a task selected from the group consisting of: tasks that support the area of attention, tasks that support the area of language, tasks that support the area of memory, tasks that support the area of problem solving, and tasks that support the area of psychomotor skills; wherein tasks that support the area of attention include searching for a hidden target object and/or finding a matching object; tasks that support the area of language include reading interactively a story and highlighting characteristics of the story, tasks that support the area of memory include remembering presented visual material and recognizing what matches or complements previously presented information, tasks that support the area of problem solving include seeing how smaller objects fit together as part of a whole, and/or finding solutions to tasks numerically, spatially, and/or linguistically; tasks that support the area of psychomotor skills include tasks that elicit finger dexterity, movements of the hands and fingers to achieve a goal.

6. Method in accordance with claim 1, wherein the exercise program is provided as an application for a computational device, such as a smart phone, a computer, and/or a tablet PC.

7. Method in accordance with claim 1, wherein the performance of the child in the tasks is compared to its previous performances.

8. Method in accordance with claim 6, wherein the performance of the child in the tasks is displayed on screen and compared to previous performances.

9. Method in accordance with claim 1, wherein the composition is to be consumed at least once per day.

10. Method in accordance with claim 1, wherein the composition is to be consumed in the morning.

11. Method in accordance with claim 1, wherein the consumption of the composition and the execution of the exercise program are no more than 3 hours apart.

12. Method in accordance with claim 1 for use in improving the cognitive abilities of children.

13. Method in accordance with claim 1 for use in improving the learning abilities of children.

14. Method in accordance with claim 1 for use in supporting brain development in children and/or for use in securing normal brain function in children.

15. Composition comprising per daily dose at least 2.2 mg Zinc, at least 2.0 mg Iron, at least 40 mg Magnesium, at least 0.2 mg Vitamin B1, at least 2.6 mg Vitamin B3, at least 0.25 mg Vitamin B6, at least 4 μg Vitamin B8, at least 12.5 mg Vitamin C, and at least 0.15 g Alpha Linolenic Acid.
